(19) United States
(12) Patent Application Publication
(10) Pub. No.: US 2018/0178025 A1
(43) Pub. Date: Jun. 28, 2018

(54) System and Method for Intracochlear and Vestibular Magnetic Stimulation

(71) Applicant: Georgia Tech Research Corporation, Atlanta, GA (US)

(72) Inventors: Sagarika Mukesh, Atlanta, GA (US); Pamela Bhatti, Atlanta, GA (US); David Blake, Atlanta, GA (US); Brian McKinnon, Atlanta, GA (US)

(21) Appl. No.: 15/854,873

(22) Filed: Dec. 27, 2017

Related U.S. Application Data
(60) Provisional application No. 62/439,216, filed on Dec. 27, 2016.

(51) Int. Cl. A61N 2/00; A61N 2/02

(52) U.S. Cl. CPC A61N 2/006; A61N 1/0541; A61N 2/02

(57) ABSTRACT

The disclosure relates to implant systems and methods for stimulation of the cochlea, auditory nerve, and/or vestibular system using an intra-cochlear magnetic stimulation electrode array that uses targeted magnetic stimulation to induce neural activation without the need for mechanical transduction. The magnetic field produced by the array stimulates portions of the cochlea or provides signals to the vestibular system.

### CROSS REFERENCE TO RELATED APPLICATIONS

This application claims priority to U.S. provisional patent application No. 62/439,216 entitled “System and Method for Intracochlear and Vestibular Magnetic Stimulation” and filed Dec. 27, 2016, the contents of which are incorporated herein by reference in their entirety as if set forth verbatim.

### STATEMENT REGARDING FEDERALLY SPONSORED RESEARCH

This disclosure was made with United States Government support from the National Institutes of Health, contract number TR000454, and the National Science Foundation, contract numbers 1133625 and 1055801. The United States Government has certain rights to this disclosure.

### FIELD OF DISCLOSURE

The present disclosure relates to medical implants, and more specifically to a novel device and method of stimulating the cochlea, auditory nerve and vestibular system.

### BACKGROUND

In a healthy human ear, sound vibrations result in resonant vibrations of the basilar membrane inside the cochlea. Differing acoustic frequencies excite corresponding sections of the cochlea which are spatially keyed whereby the outermost sections of the cochlea correspond to higher frequencies and the innermost sections correspond to lower frequency sounds. These vibrations normally result in movement of hair cells located along the basilar membrane resulting in nerve cell stimulation. The auditory nerve sends signals to the brain corresponding to the section of the cochlea stimulated, which determines what sound frequency the brain identifies as being heard.

Sensorineural hearing loss often results from fewer hair cells or damage to the hair cells thereby preventing the auditory nerve from receiving a signal as there are fewer or no hair cells to receive the vibrational energy. Cochlear implants are used to provide a sense of hearing to patients suffering from sensorineural hearing loss through direct stimulation of the auditory nerve to send signals to the brain.

Existing cochlear implants use an external portion which uses one or more microphones to receive sound from the environment. Sounds which are received are then translated using a processor which filters sound signals into information which is then conveyed to an internal portion, either through electrical impulses carried through a cable or electromagnetic induction through tissue.

The internal portion of the cochlear implant receives signals conveyed from the external portion and sends signals to the auditory nerve through a stimulator. The signals conveyed are then translated into electric impulses which are sent through an array of electrodes wound through the cochlea. The electrodes are usually placed in the scala tympani and exciting them causes signals to be sent to the brain through the auditory nerve.

Cochlear implants use electrodes inserted into the cochlea to stimulate those portions of the cochlea for which acoustic frequencies are not perceived. Electrodes used in such implants are platinum, iridium, or titanium. Currents from tens to hundreds of micro amps are applied to the electrodes to stimulate the cochlea. The electrodes are inserted into the scala vestibuli or tympani and are bathed in perilymph, which is a saline solution found in the cochlea. In various cochlear implants, the electrodes are in contact with tissue. Such contact can result in formation of scar tissue (e.g., fibrosis) around the electrode.

Cochlear implant stimuli may be monopolar. This means that current generated at the electrodes is spread on its path to the ground, which is typically near the oval or round window that exits to the cochlea. Cochlear implant stimuli may also be bipolar, with one electrode serving as a source and a nearby electrode serving as a sink.

The function of cochlear implants is limited by the number of effective frequency channel they can stimulate. Although implants can have 22 or more electrodes in a 17 mm implant length, they typically achieve no more than six effective frequency channels. Current spread from existing electrodes is one potential limitation on that effective limit. Performance is limited by the number of effective channels stimulated, and also by the necessity of adjusting stimuli for each electrical contact based on efficacy in eliciting a response. The efficacy varies as a function of distance from the modiolus, tissue response, and the tissue-electrode impedance.

This method of stimulating the auditory nerve suffers from poor sound quality in recipients of the treatment and also suffers sound degradation over time. Sending electric currents to convey signal to the auditory nerve can have added difficulty as it requires the current to travel through fluid and tissue which can adversely impact the precision of the signal being delivered to the auditory nerve through attenuation of the signal which when coupled with the current spread results in suboptimal results. As the body adapts to the presence of a foreign object by forming scar tissue, fibrosis causes the development of more tissue which further exacerbates attenuation before reaching the modiolus.

Current cochlear implants require the use of expensive metals resulting from the need to be in contact with human tissue in the recipient's body, which leads to a more expensive product which can prevent a sizeable number of potential recipients from affording the technology.

Vertigo is a disorder which results in the subject experiencing the sensation of movement, often independent of external stimuli. Semicircular canal system dysfunction can cause inaccurate or irregular signals to be produced by the lateral, superior or inferior canals' push-pull systems which then sends those signals to the vestibular nerve. These inaccurate or irregular signals can cause objects, surroundings, or the person themselves to feel as if they are moving independent of external stimuli, which often results in the sensation of spinning or swaying. This condition can result in vomiting, nausea, imbalance, and other adverse effects.

Current treatments of vertigo include prescribing medications such as prochlorperazine for balance or antihistamines which are often prescribed to treat motion sickness or nausea. Although these treatments can be effective for some patients, the results vary and are often imprecise or unpredictable which can result in unwanted side effects. These medications are often used to treat symptoms of vertigo reactively instead of preventing the onset of symptoms. Physical therapy and rehabilitation may also be used as treatment methods, but again this method of treatment provides only for a reaction to the onset of symptoms in hopes of preventing future recurrences.

There is a need for a better method of stimulating auditory neurons so that a greater number of frequencies can be stimulated with greater precision and circumventing attenuation from using tissue as a carrier for signals sent to those auditory neurons. The same solution can be used to circumvent the problems facing treatments for vertigo stemming from semi-circular canal dysfunction whereby the current treatments can be supplanted with a permanent implant that produces predictable results and prevents occurrences of vertigo.

### SUMMARY

Scientists have been actively studying magnetic stimulation of excitable tissue and have seen some success in different applications. Clinically, transcranial magnetic stimulation (TMS) is a non-invasive method employed for diagnostic and therapeutic purposes to treat depression, migraine, as well as improve motor signals in those suffering from Parkinson's disease.

The present disclosure relates to the stimulation of the cochlea, auditory nerve, and vestibular system using an implantable magnetic stimulation array that uses targeted magnetic stimulation to induce neural activation without the need for mechanical transduction. The implantable magnetic stimulation array may be intra-cochlear, e.g., implanted into the cochlea or configured to stimulate neurons primarily in the cochlea. The implantable stimulation array may be vestibular, e.g., implanted in a manner, or configured to, stimulate neurons primarily in the vestibular system. In the case of the cochlea, this intra-cochlear magnetic stimulation array produces electrical fields created by a miniature coil of wire that is easily inserted into a cochlea that produces an electrical field with a rate of change that produces a magnetic field. The magnetic field can be time-varying. The resultant magnetic field is then used to stimulate portions of the cochlea or provide signals. A similar scheme may be applied to the target sensory organs in the peripheral vestibular system; namely the semicircular canals and the otolith.

The present disclosure comprises an implantable array and method of stimulating the cochlea and vestibular system using targeted magnetic fields. The device produces a time-varying electric current in the form of pulses which is passed through inductors thereby inducing a time-varying magnetic field both in the inductor and the surrounding tissue. The magnetic fields then generate a sufficient potential difference to excite neurons and provide excitation of peripheral processes without mechanical transduction. This method is used in an intra-cochlear magnetic stimulation array which produces the aforementioned magnetic fields through pulsatile current stimulation of submillimeter inductors. The array will be designed for insertion into a human cochlea.

In one aspect is provided cochlear implant system, an implant system for stimulating a cochlea and/or a vestibular system is disclosed. The system can include a loop (e.g., a coil, a loop of wire, a metal loop) comprising a first end and a second end, a wire comprising a first end connected to the first end of the loop and a second end connected to the second end of the loop, and an electrode array disposed on or in communication with the wire, the electrode array adapted to be inserted into the cochlea. The first end of the wire receives electrical impulses representing recorded signals and conveys the electrical impulses through the loop towards the second end of the wire causing a time-varying magnetic field to be induced in the loop that creates a transient electrical field capable of stimulating the cochlea and/or vestibular system.

In some embodiments, the system can include an insulator that encapsulates the loop. The insulator can be biocompatible polymer that encapsulates the loop from between the first and second ends of the loop. The loop can be a piece of metallic wire that is an inductive coil comprising or in communication with the electrode array.

In some embodiments, stimulation of the cochlea by the transient electrical field causes an auditory brainstem response. The auditory brainstem response can be indicative of a frequency of the sound signals represented by the received electrical impulses.

In some embodiments, stimulation of the vestibular system by the transient electrical field causes a vestibular nerve response. The vestibular nerve response can be indicative of spatial and orientation information of the signals represented by the received electrical impulses.

In some embodiments, a silicone plug is included that surrounds a coil connected to or in communication with the loop to facilitate insertion of the loop inside the cochlea.

In another aspect is provided a method for stimulating a cochlea, comprising (a) receiving electrical impulses representing sound signals, (b) conveying the electrical impulses to an inductive coil inserted within the cochlea, and (c) inducing a time-varying magnetic field in the coil in response to the electrical impulses to create a transient electrical field that stimulates the cochlea.

In some embodiments, a method for stimulating a cochlea and/or a vestibular system is also disclosed. The method can include delivering an implant system to the cochlea. The implant system can include an electrode array adapted to be inserted into the cochlea; a loop (e.g., a coil, a loop of wire, a metal loop) comprising a first end and a second end; a wire comprising a first end connected to the first end of the loop; and a second end connected to the second end of the loop. The method can include the implant system receiving electrical impulses representing sound signals; conveying the electrical impulses through the loop to the electrode array inserted within the cochlea; and inducing a time-varying magnetic field in the loop in response to the electrical impulses to create a transient electrical field that stimulates the cochlea and/or the vestibular system.

In some embodiments, the method can include stimulating the cochlea and/or the vestibular system by the transient electrical field to cause an auditory brainstem response and/or a vestibular nerve response.

In some embodiments, the auditory brainstem response is indicative of a frequency of the sound signals represented by the received electrical impulses.

In some embodiments, the vestibular nerve response is indicative of spatial and orientation information of the signals represented by the received electrical impulses.

In some embodiments, stimulation of the cochlea and/or the vestibular system by the transient electrical field varies depending upon the strength and timing of the received electrical impulses.

In some embodiments, the method can include arranging the loop so as to not be in direct contact with tissue of the cochlea.

In some embodiments, a method of fabricating a cochlear implant system is also disclosed. The method can include forming a substrate with a flexible material; printing metal traces on the substrate using conductive ink to form an array of coils; separating each coil by a predetermined distance; positioning insulating bridge material over the array of coils; and encapsulating the substrate and the array of coils in a biocompatible polymer.

In some embodiments, the flexible material is polyimide or Kapton.

In some embodiments, the conductive ink is aluminum, silver, gold or copper.

In some embodiments, the step of printing is 3D printing.

In some embodiments, the predetermined distance is 5 mm. However, the distance can be smaller or larger, as needed or required.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts a conventional cochlear implant system implanted inside a human cochlea consisting of a microphone, magnetic transmitter/receiver and an electrode array while not necessarily depicting all other external components.

FIG. 2 depicts an example implant system of this disclosure having an array of implanted inductors in close proximity to target neurons (within predetermined dimension of approximately 0.5 mm).

FIG. 3 depicts a close-up view of section A-A from FIG. 2.

FIG. 4 depicts a schematic overview of an example method for stimulating the cochlea and/or vestibular system.

FIG. 5 a schematic overview of an example method for stimulating the cochlea and/or vestibular system.

FIG. 6 a schematic overview of an example method for fabricating a cochlear implant system.

FIG. 7 depicts a graph showing the electric field amplitude [V/m] as a function of perpendicular distance from the surface of the coil [micrometers].

FIG. 8 depicts a view looking down on an example coil used with a loop of one example implant system of this disclosure.

### DETAILED DESCRIPTION OF THE DISCLOSURE

Detailed embodiments of the present disclosure are disclosed herein; however, it is to be understood that the disclosed embodiments are merely illustrative of the disclosure that may be embodied in various forms. In addition, each of the examples given in connection with the various embodiments of the disclosure is intended to be illustrative, and not restrictive. Therefore, specific structural and functional details disclosed herein are not to be interpreted as limiting, but merely as a representative basis for teaching one skilled in the art to variously employ the present disclosure.

Additionally, it has been contemplated that the claimed devices, systems and methods can be embodied in other ways, to include different steps or elements similar to the ones described in this document, in conjunction with other present or future technologies. Although the term “step” can be used herein to connote different aspects of methods employed, the term should not be interpreted as implying any particular order among or between various steps herein disclosed unless and except when the order of individual steps is explicitly required. To facilitate an understanding of the principles and features of the present disclosure, embodiments are explained hereinafter with reference to implementation in illustrative embodiments. The system of this disclosure can include an implantable array which will interface or communicate with other systems which may include external microphones, speech processors, transmitters and implanted receivers among others. These external systems will either record signals to be sent to auditory neurons or otherwise provide what signals the internal array should produce and to what area those signals should be sent.

Unless defined otherwise, all technical and scientific terms used herein have the same meaning as commonly understood by one of ordinary skill in the art to which this disclosure belongs.

It is to be understood that this disclosure is not limited to the specific devices, methods, conditions, or parameters described and/or shown herein, and that the terminology used herein is for the purpose of describing particular embodiments by way of example only. Thus, the terminology is intended to be broadly construed and is not intended to be limiting of the claimed disclosure. For example, as used in the specification including the appended claims, the singular forms “a,” “an,” and “one” include the plural, the term “or” means “and/or,” and reference to a particular numerical value includes at least that particular value, unless the context clearly dictates otherwise. In addition, any methods described herein are not intended to be limited to the sequence of steps described but can be carried out in other sequences, unless expressly stated otherwise herein.

Ranges can be expressed herein as from “about” or “approximately” one particular value and/or to “about” or “approximately” another particular value. When such a range is expressed, other exemplary embodiments include from the one particular value and/or to the other particular value. The terms “comprising” or “containing” or “including” mean that at least the named component, element, particle, or method step is present in the system or article or method, but does not exclude the presence of other components, materials, particles, or method steps, even if the other such components, material, particles, and method steps have the same function as what is named.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

A “subject” or “patient” or “individual” or “animal”, as used herein, refers to humans, veterinary animals (e.g., cats, dogs, cows, horses, sheep, pigs, etc.) and experimental animal models of diseases (e.g., mice, rats). In a preferred embodiment, the subject is a human.

The terms “antenna” and “loop”, as used herein, are interchangeable.

For users who fail to gain the intended benefits from existing cochlear implant technologies, causes for such failure may range from implantation technique and depth, distance from, and concentration of, the target spiral ganglion cells housed in the central modiolus, and/or spread of electrical excitation current. Further, the central auditory system and high-level central associative areas play an equally significant role. In contrast, factors that affect success of existing cochlear implant technologies include age of implantation, duration of auditory deprivation, and an individual's brain plasticity. And while prior approaches have advanced sound coding and signal processing strategies to dramatically improve the transmission of sound information as well as pioneered current shaping, the addressable space within the peripheral auditory system remains a constant challenge. Further, the distance from cochlear implant stimulation sites to spiral ganglion neurons, as well as the poor survival of spiral ganglion neurons, result in sub-optimal coupling between the device and the cochlea. This is further aggravated by the inability to precisely control generated fields due to the highly electrically conductive intracochlear fluid resulting in poor frequency specificity, as well as increased channel interaction (interference). Furthermore, fibrous encapsulation of the implanted array serves to further diminish control of injected charge. These shortcomings are associated with poor speech understanding. Additionally, for individuals with limited neural survival where the addressable neural space is sparse, the lack of specificity dramatically limits cochlear implant benefit.

To improve coupling to the neural space, arrays can be coiled closely to the central modiolus with mixed outcomes indicating that the microvasculature in the cochlea is adversely affected by this placement. And finally, postmortem studies have shown that metal loss occurs due to charge imbalance during stimulation causing metal migration over time to adjacent tissue, compromising the long-term efficacy of electrode-based stimulation. When considering the need for implantation in children and the increasing rate of early implantation, long-term efficacy is critical.

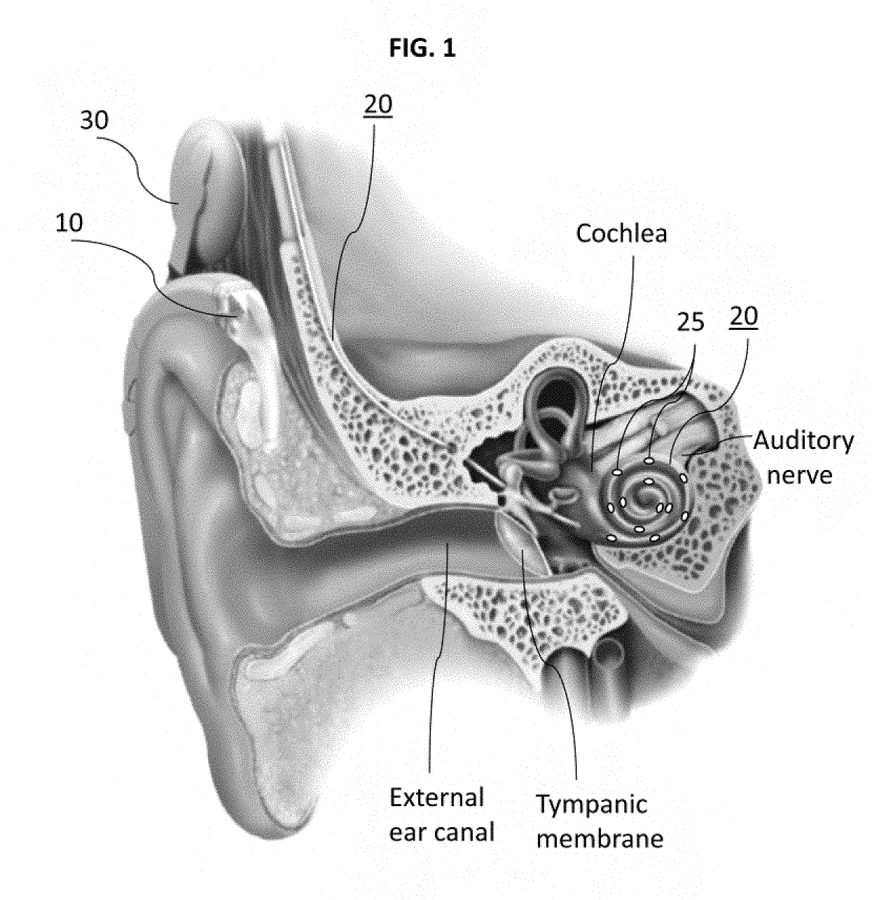

FIG. 1 depicts a conventional cochlear implant system 10. The system can include an external component 10 (e.g., a microphone, telecoils, sound processing unit, external coil, etc.). The external component 10 can be connected to the patient. The system 10 can also include a loop 30 in communication with a wire 20. The implantable portions of the system of FIG. 1 can include the wire 20, and an electrode array 25. Wire 20 can extend from loop 30 with an electrode array 25 disposed in communication with the auditory nerve and implanted in the cochlea. Wire 20 can be flexible so that during delivery into the cochlea it can assume a tortuous shape of the cochlea. Though not depicted, the system can also include a magnet or a plurality of magnets. The magnet(s) can facilitate the operational alignment coils, externally and those implanted.

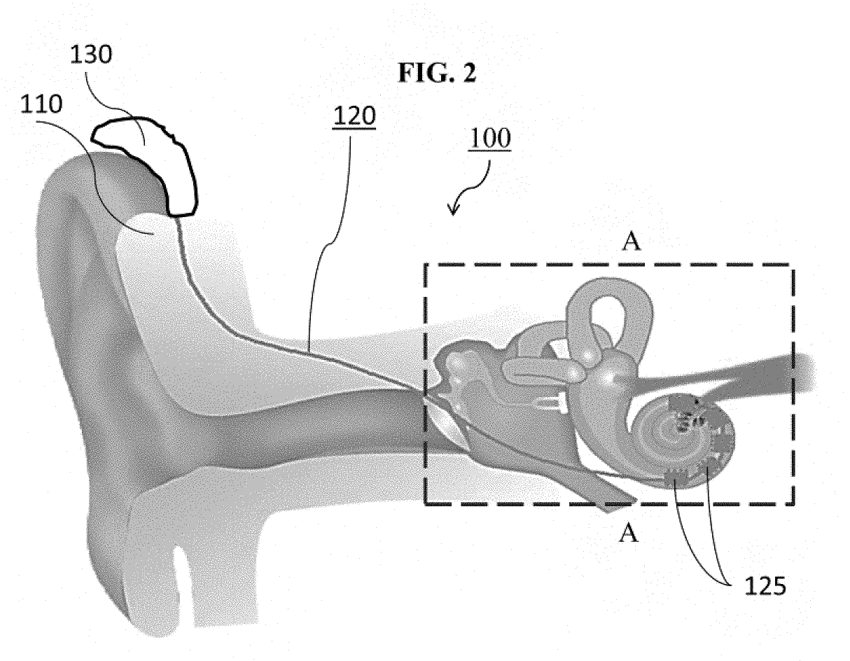

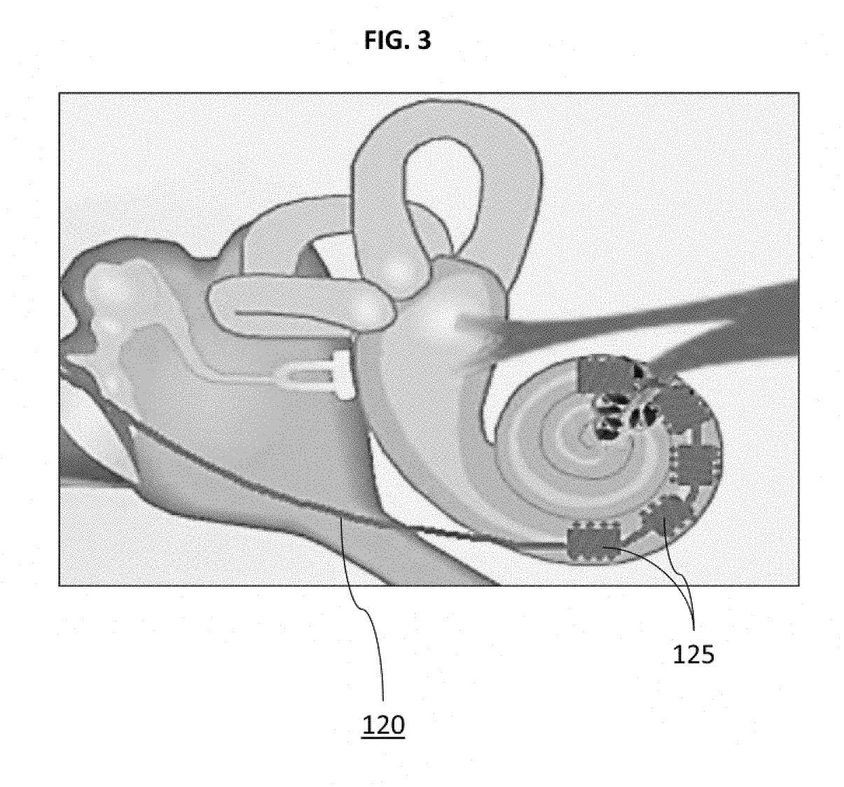

Turning to FIG. 2, one embodiment of the herein disclosed magnetic stimulation system 100 is illustrated having an array of implanted inductors 125. The system 100 can include a microphone 110 in communication with a loop 130. Wire 120 can be part of the antenna and/or can extend from a first end of the antenna and into the cochlea. In this respect, the loop 130 can comprise an electrode array 125 implantable in the cochlea. This is more clearly seen in FIG. 3 which shows a close-up view of section A-A of FIG. 2. The electrode array 125 is arranged with loop 130 and wire 120 and is capable of inducing electric fields for excitation capability when implanted with the cochlea. In certain embodiments, the inductors 125 of system 100 are in close proximity to the target neurons (within 0.5 mm). The intention of this sketch is to convey the position where these inductors are placed and not necessarily the distance between the inductors or their number. For example, while 0.5 mm may be a preferred dimension of separation, any dimension can be used as needed or required. The external components (e.g., microphone 110) as well as implanted driving circuitry are not shown in this view.

In one aspect is provided an implant system for the cochlea and/or the vestibular system. The system can include an electrode array, a loop having a first end and a second end, and a wire having a first end of the wire connected to the first end of the loop and a second end of the wire connected to the second end of the loop. The electrode array of the cochlear implant system is adapted to be inserted into the cochlea such that the first end of the wire receives electrical impulses representing recorded signals and conveys the electrical impulses through the loop towards the second end, causing a time-varying magnetic field to be induced in the loop which creates a transient electrical field that stimulates the cochlea, vestibular system, or both. The loop can be an inductive coil and/or include the electrode array of the system.

In some embodiments, the electrode array comprises metal electrodes. In some embodiments, the electrode array with metal electrodes of the implant system can be replaced with one or more microcoils. The system in this respect could be used with peripheral sensory organs such as the cochlea, vestibular system, retina, as well as central applications in deep brain stimulation. In addition, the system of this disclosure is contemplated for use as with dual electrode-and-coil arrays for situations where both modalities are efficacious.

In some embodiments, the cochlear implant system further can include an insulator that encapsulates the loop. The insulator may be a biocompatible polymer, such as Parlyene-C or polyimide. The biocompatible polymer can also be silicone (e.g., PDMS) or a liquid crystal polymer. The insulator may comprise Parylene-C. The insulator may comprise polyimide.

The cochlear implant system may be configured to deliver varying field strengths. The field strength may range from 1 to 20 V/m, from 3 to 18 V/m, from 6 to 15 V/m, from 8 to 12 V/m, from 1 to 3 V/m, from 2 to 4 V/m, from 3 to 5 V/m, from 4 to 6 V/m, from 5 to 7 V/m, from 6 to 8 V/m, from 7 to 9 V/m, from 8 to 10 V/m, from 9 to 11 V/m, from 10 to 12 V/m, from 11 to 13 V/m, from 12 to 14 V/m, from 13 to 15 V/m, from 14 to 16 V/m, from 15 to 17 V/m, from 16 to 18 V/m, from 17 to 19 V/m, or from 18 to 20 V/m.

The insulator may be effective to prevent direct contact between bodily tissues and fluids and one or more of the other components of the cochlear implant system that is metallic or generates an electric field, e.g., a wire, a coil, etc. The insulator also may not substantially affect the field penetration. Without wishing to be bound by theory, the lack of direct contact between bodily fluids and metallic components or direct sources of electric field can minimize scarring and formation of fibroses. Prevention, minimization or reduction in the rate of fibrosis formation can prevent loss of performance of cochlear implant electrodes and increase of system power consumption.

In some embodiments, the loop is a piece of metallic wire that is an inductive coil. The inductors may be a 0.5 mm×0.5 mm quartz core with 15-25, 16-22, 17-23, 18-24, 18-22, 18, 19, 20, 21, or 22 turns of copper coil. Other cores and other metals may be used by those having ordinary skill in the art. The inductors can have various inductances. For example, the inductance can range from 500 nH to 3.5 mH, from 600 nH to 3.2 mH, from 650 nH to 3.0 mH, from 680 nH to 2.8 mH, from 800 nH to 2.5 mH, from 1.0 mH to 2.3 mH, from 1.2 mH to 2.0 mH, from 1.4 mH to 1.8 mH, from 1.6 mH to 1.7 mH, from 500 nH to 700 nH, from 600 nH to 800 nH, from 700 nH to 900 nH, from 800 nH to 1000 nH, from 900 nH to 1100 nH, from 1.0 mH to 1.2 mH, from 1.1 mH to 1.3 mH, from 1.2 mH to 1.4 mH, from 1.3 mH to 1.5 mH, from 1.4 mH to 1.6 mH, from 1.5 mH to 1.7 mH, from 1.6 mH to 1.8 mH, from 1.7 mH to 1.9 mH, from 1.8 mH to 2.0 mH, from 1.9 mH to 2.1 mH, from 2.0 mH to 2.2 mH, from 2.1 mH to 2.3 mH, from 2.2 mH to 2.4 mH, from 2.3 mH to 2.5 mH, from 2.4 mH to 2.6 mH, from 2.5 mH to 2.7 mH, from 2.6 mH to 2.8 mH, from 2.7 mH to 2.9 mH, from 2.8 mH to 3.0 mH, from 2.9 mH to 3.1 mH, from 3.0 mH to 3.2 mH, from 3.1 mH to 3.3 mH, from 3.2 mH to 3.4 mH, or from 3.3 mH to 3.5 mH.

The loop may comprise multiple coils. The multiple coils may be configured as an array. The configuration of the array can provide for additional field focusing.

In some embodiments, stimulation of the cochlea by the transient electrical field causes an auditory brainstem response. In some embodiments, stimulation of the cochlea by the transient electrical field varies depending upon the strength and timing of the received electrical impulses. In some embodiments, the auditory brainstem response is indicative of a frequency of the sound signals represented by the received electrical impulses. In some embodiments, the coil is not in direct contact with tissue of the cochlea. The cochlear implant system may be configured such that the coil is perpendicular to the surrounding tissue surface. The cochlear implant system may be configured such that the coil is parallel to the surrounding tissue surface. The cochlear implant system may be configured such that the coil is at least 60 μm, at least 70 μm, at least 80 μm, at least 90 μm, at least 100 μm, at least 110 μm, or at least 120 μm from the inside of the cochlea.

In some embodiments, the stimulation of the vestibular system by the transient electrical field causes a vestibular nerve response. In some embodiments, the stimulation of the vestibular system by the transient electrical field varies depending on the strength and timing of the received electrical impulses. In some embodiments, the vestibular nerve response is a firing rate that is indicative of head motion frequency. In some embodiments, the vestibular nerve response is indicative of spatial and orientation information of the signals represented by the received electrical impulses. In some embodiments, the cochlear implant system further comprises a silicone plug surrounding the coil to facilitate insertion of the coil inside the cochlea.

The device comprising an intra-cochlear magnetic stimulation array can be inserted into the scala tympani and produce a current pulse representing a sound impulse or orientation signal. An example can be a coil of wire 1 mm long and 0.8 mm wide which may be encased in a plug to facilitate insertion into the cochlea. The plug may be made of silicone or other materials to facilitate its implantation into the cochlea and illustrates that the array is not designed come into contact with cochlear tissue and does not produce direct electrical stimulation of the cochlea.

The current pulse is introduced along a wire at one end of the coil and runs through the coil towards the other end of the coil. This current pulse represents either sound or orientation signals received by communications from an external device. This external device record sound through a microphone, orientation through mechanisms such as an accelerometer, or both. These recordings are then communicated to an external or internal signal processor which interprets the recorded information into sound frequency or orientation signal.

The current pulse induces a magnetic field that runs axially to the coil and produces a transient electrical field that travels transversely to the magnetic array's wire and inversely to the magnetic field and around the circumference of the coil.

One benefit of this targeted magnetic stimulation is that it will provide a sense of hearing to patients suffering from sensorineural hearing loss by exciting the spatial areas currently not receiving stimulation through sound vibrations. Magnetic fields couple with auditory neurons to produce excitation that is perceived by the brain as a sound signal corresponding to a frequency within the range of 20-20 k Hz and do so with a more precise frequency resolution which in turns produces better sound quality.

Another benefit of this targeted magnetic stimulation is that the intervening tissue between the array and the auditory neurons is no longer a carrier for the signals which reduces attenuation suffered by the current methods of delivering signals.

Yet another benefit of the targeted magnetic stimulation is that tissue build up resulting from fibrosis no longer affects signal delivery to the same degree as the current methods. This produces a more reliable signal with decreased sound quality degradation over time.

Materials utilized for production of current cochlear implants that use direct electrical stimulation are expensive metals due to contact with human tissue. Another benefit to targeted magnetic stimulation is that since there is not direct electrical stimulation, a greater range of materials can be used to produce the implantable magnetic array. This may result in more efficient or cost effective methods of production.

In stimulating the vestibular system, targeting magnetic stimulation can send signals to supplant the function of the semi-circular canal systems which can prevent the onset of vertigo. This is done by reading the axis by which the head and body are currently held and sending that signal to neurons which in turn allows circumvention of semi-circular canal system dysfunction from causing vertigo in the recipient of the treatment.

One embodiment allows for the electrical impulse to produce a bipolar electric field. This allows for increased frequency resolution by increasing the number of frequency channels perceived by the cochlea or improved orientation signal perceived by a vestibular nerve or neuron. This can drastically increase the frequency channels communicated or correct dysfunctional semicircular canal signals.

For magnetic stimulation, the induced electric field amplitude [V/m] is a performance characteristic. In contrast, the electrostatic potential [V] is the performance characteristic for prior art implants that use electrical stimulation. See Mukesh, S. et al., IEEE Transactions on Neural Systems and Rehabilitation Engineering, 2017, 25(8): 1353-1362. In certain embodiments, the induced electric fields of the system can be less than 10V/m and activate the neural tissue.

Electric fields produced by magnetic coils do not attenuate significantly up to a depth of 100 μm into the modiolus. This implies a potential for direct stimulation of the cochlear nerve, in case the peripheral dendrites are completely damaged, is possible. Typically, the fields induced in the cochlea are up to 10 times stronger when the axis of the coil was placed parallel to the modiolus, as opposed to when the axis is placed perpendicular to it. An array of these inductors shows negligible degrees of cross talk and highly focused stimulation (spatial resolution of 100 μm).

The electric field may alternatively be produced using a simple copper wire shaped to work as a dipole loop. Although any geometrical configuration may be used, a quadrilateral design is the most common shape used. An array of such dipoles function to increase frequency resolution compared to the implants presently used while consuming lower power than other magnetic array designs. This dipole design has the potential to focus the electric field better in the desired region and has traditionally shown lower power consumption.

In another variation of employing the method described, the proposed magnetic array may be combined with traditional electrical stimulation. Despite the drawbacks of current technology, its simplicity and effectiveness still serve beneficial treatment effects. It may be situationally beneficial to combine the novel magnetic stimulation with existent electric stimulation methods.

The proposed circuit that will be used to drive the above-mentioned arrays is within the FDA limits of permissible power usage in and around the human body. This circuit will be uniquely designed to be compatible with the above described arrays.

The selectivity of the stimulation allows for a collection of improvements which culminate into the largest improvement in cochlear implant function in decades. Targeted magnetic stimulation improves frequency resolution which in turn improves the sound quality perceived by users. Avoiding the use of direct electric stimulation decreases attenuation and mitigates the advancement of sound degradation resultant from fibrosis. Since the inductor is fully insulated from tissue, tissue reaction should be minimized. The use of targeted magnetic stimulation can excite neurons to provide a sense of hearing to those suffering from hearing loss or the cessation of vertigo without having to employ multiple devices. Lastly, by using a method other than direct electrical stimulation a wider range of production materials may be utilized, which may drastically reduce production costs.

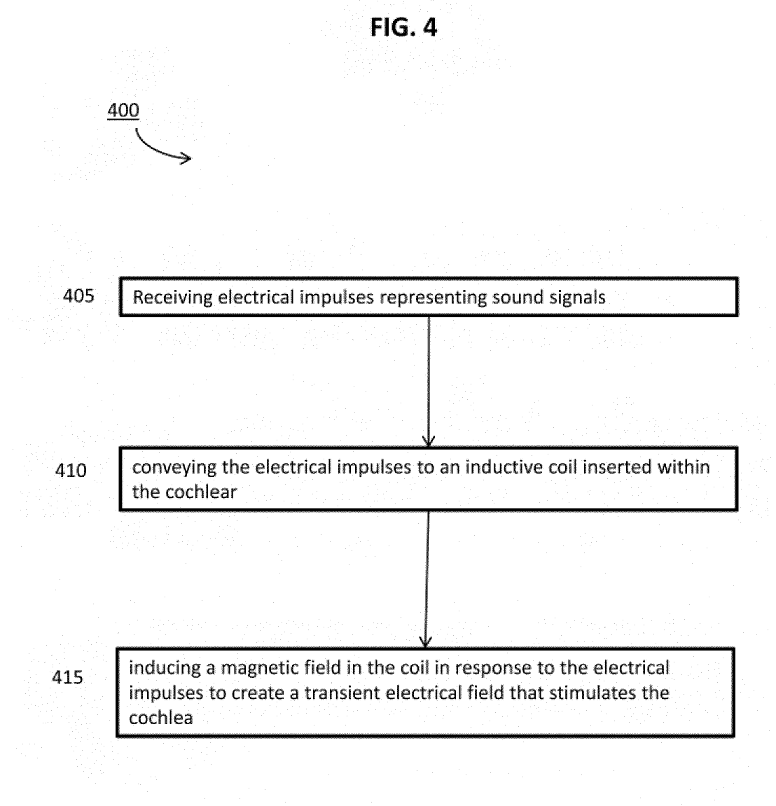

In FIG. 4, a method 400 is disclosed for stimulating a cochlea and/or vestibular system. The method can include 405 receiving electrical impulses representing sound signals, 410 conveying the electrical impulses to an inductive coil inserted within the cochlea, and 415 inducing a time-varying magnetic field in the coil in response to the electrical impulses to create a transient electrical field that stimulates the cochlea.

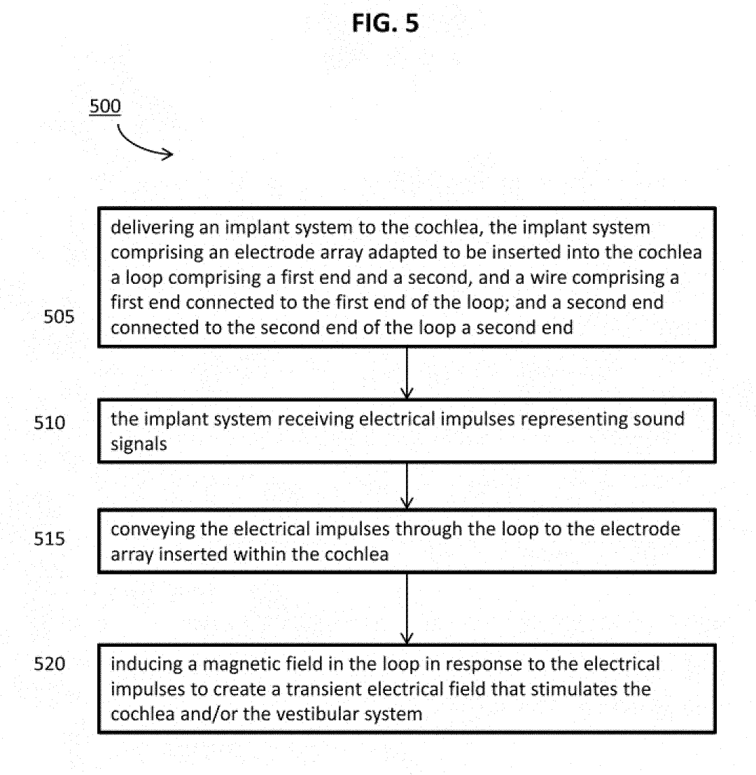

In FIG. 5, a method 500 is disclosed for stimulating a cochlea and/or vestibular system. The method can include 505 delivering an implant system of this disclosure to the cochlea. Accordingly, the implant system can include an electrode array adapted to be inserted into the cochlea a loop comprising a first end and a second, and a wire comprising a first end connected to the first end of the loop; and a second end connected to the second end of the loop a second end. Step 510 can include the implant system receiving electrical impulses representing sound signals. Step 515 can include conveying the electrical impulses through the loop to the electrode array inserted within the cochlea. Step 520 can include inducing a time-varying magnetic field in the loop in response to the electrical impulses to create a transient electrical field that stimulates the cochlea and/or the vestibular system.

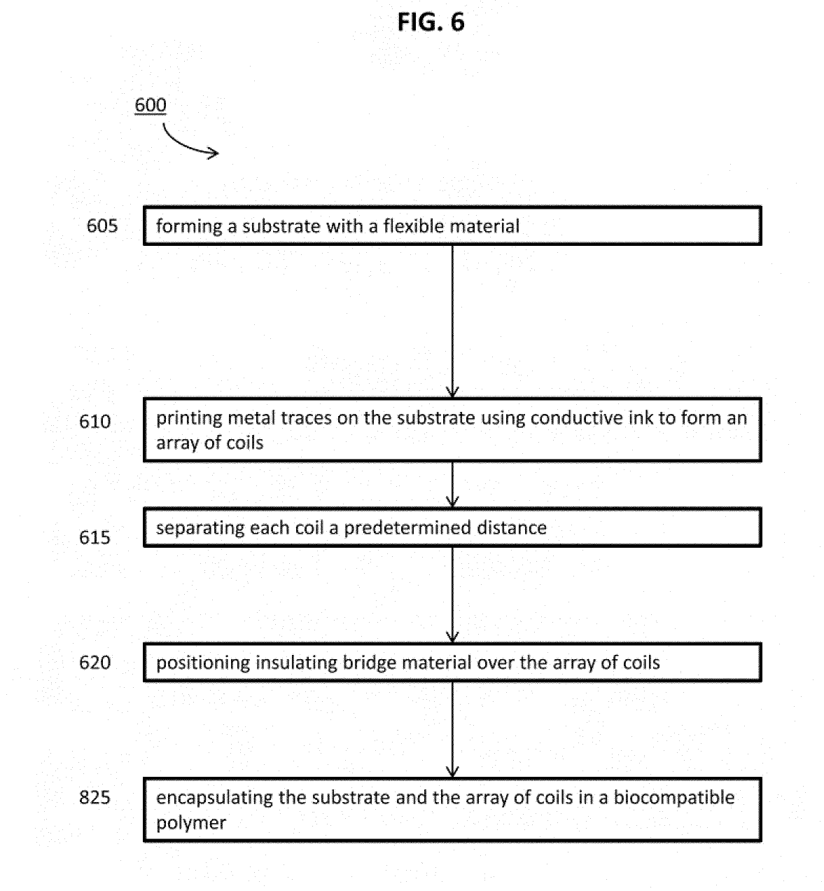

In FIG. 6, a method 600 of fabricating a cochlear implant system is depicted. Step 605 can include forming a substrate with a flexible material. Step 610 can include printing metal traces on the substrate using conductive ink to form an array of coils. Step 615 can include separating each coil by a predetermined distance. Step 620 can include positioning insulating bridge material over the array of coils. Step 625 can include encapsulating the substrate and the array of coils in a biocompatible polymer.

This and other methods of fabricating devices described herein can overcome the current cost disadvantages of making cochlear implant devices by hand.

In another aspect is provided a method for stimulating a cochlea, comprising (a) receiving electrical impulses representing sound signals, (b) conveying the electrical impulses to a loop or an inductive coil inserted within a cochlear implant device described herein, and (c) inducing a magnetic field in the loop or coil in response to the electrical impulses to create a transient electrical field that stimulates the cochlea.

In another aspect is provided a method for treating a patient or subject suffering from profound hearing loss comprising (a) receiving electrical impulses representing sound signals, (b) conveying the electrical impulses to a loop or an inductive coil inserted within a cochlear implant device described herein, and (c) inducing a magnetic field in the loop or coil in response to the electrical impulses to create a transient electrical field that stimulates the cochlea. The device is effective to ameliorate or treat the profound hearing loss, e.g., by providing some improvement in the ability of the patient or subject to hear sounds.

In another aspect is provided a method for treating a patient suffering from bilateral vestibular dysfunction comprising (a) receiving electrical impulses from an external transmitter and/or an external sensor, (b) conveying the electrical impulses to an inductive coil inserted within a vestibular implant device described herein, and (c) inducing a magnetic field in the coil in response to the electrical impulses to create a transient electrical field that stimulates the vestibular system. The external transmitter may generate electrical impulses based on a program, e.g., a pacemaker application. In some embodiments, an external sensor is used to measure head rotations and movements, with the external transmitter generating electrical impulses based on such head rotations and movements. An exemplary external sensor is part of an implantable vestibular prosthesis, as described in Toreyin, H. and Bhatti, P., “A Low-Power ASIC Signal Processor for a Vestibular Prosthesis” IEEE Transactions on Biomedical Circuits and Systems, 2016, Vol. 10, No. 3, incorporated herein by reference in its entirety as if set forth verbatim.

An implantable vestibular prosthesis can sense angular and linear head motions through angular velocity sensors and/or linear acceleration sensors. Such angular velocity sensors and linear acceleration sensors may be commercial. The implantable vestibular prosthesis is configured to 1) align implanted and natural inertial sensors and optimize stimulation efficacy, 2) mimic the neural dynamics of the vestibular system, and 3) generate drive signals for neural stimulators that modulate the neural firing rate of vestibular neurons.

The peripheral vestibular system may comprise three SCCs and two otoliths, each having a direction of maximum sensitivity. Following the surgical implantation of a VP, a coordinate system transformation, implemented as a vector-matrix multiplication, can be used to align the implanted inertial sensors with the natural SCC and otolith. The vector-matrix multiplication can also serve to improve stimulation efficacy. For instance, when an electrode is placed to stimulate the horizontal canal neurons, some posterior canal neurons may be falsely stimulated.

In some embodiments, the stimulation of the vestibular system by the transient electrical field causes a vestibular nerve response. In some embodiments, the stimulation of the vestibular system by the transient electrical field varies depending on the strength and timing of the received electrical impulses. In some embodiments, the vestibular nerve response is indicative of spatial and orientation information of the signals represented by the received electrical impulses.

In stimulating the vestibular system, the targeted magnetic stimulation can send signals to supplant the function of the semi-circular canal systems which can prevent the onset of vertigo. Signals may be generated based on a reading the axis by which the head and body are currently held. The signals may be transmitted to neurons by the vestibular implant device, allowing circumvention of semi-circular canal system dysfunction from causing vertigo in the recipient of the treatment.

The vestibular system includes the parts of the inner ear and brain that process the sensory information involved with controlling balance and eye movements. Impairment of the vestibular system can lead to a number of symptoms including difficulty maintaining balance, sensation of vertigo, sensation of being out of balance not appropriate to the situation (e.g., when moving but not in a situation where a person with normal vestibular system functioning would sense lack of balance), and strange sensations accompanying head movement. Various vestibular and cochlear implant devices described herein may be effective to improve functioning of the vestibular system, ameliorate or treat impairments of the vestibular system. Stimulation of various neurons in the inner ear by the cochlear implant devices describe herein may restore ability to maintain balance, reduce or eliminate sensations of vertigo, reduce or eliminate sensations of being out of balance, and/or reduce or eliminate any strange sensations accompanying head movement.

While the disclosure has been shown and described in exemplary forms, it will be apparent to those skilled in the art that many modifications, additions, and deletions can be made therein without departing from the spirit and scope of the disclosure as defined by the following claims. As such, the microelectromechanical techniques (MEMS) used to fabricate the arrays are designed for application to the auditory and vestibular systems, but may find use in other micro-scale applications such as retinal implants and deep brain stimulation.

### EXAMPLES

### Example 1: Fabrication of the Coil by 3-D Printing

Various aspects of the disclosed devices and methods may be still more fully understood from the following description of some example implementations and corresponding results. Some experimental data is presented herein for purposes of illustration and should not be construed as limiting the scope of the disclosed technology in any way or excluding any alternative or additional embodiments.

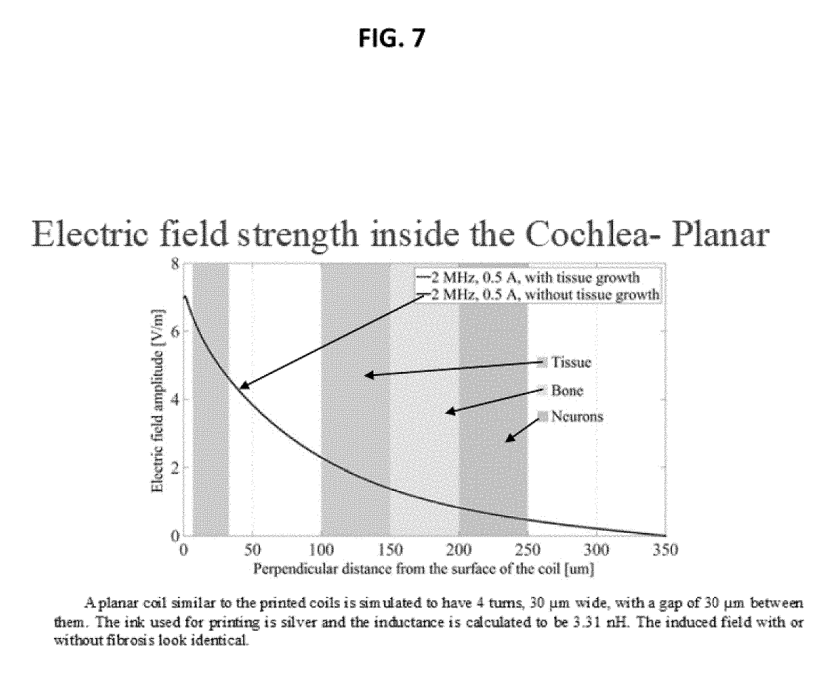

FIG. 7 depicts graphical results of microcoils of an example implant system of this disclosure when fully encapsulated with biocompatible polymers such as Parlyene-C or polyimide. There is an arrow extended through a 2 MHz signal with 0.5 A applied without tissue growth and is superimposed on another line that indicates a 2 MHz signal with 0.5 A applied with tissue growth. A planar coil similar to the printed coils of certain embodiments of the implant system of this disclosure is simulated to have 4 turns, is 30 μm wide, and has a gap of 30 μm between the coils. The ink used for printing is silver and the inductance is calculated to be 3.31 nH. The induced fields, with or without fibrosis, appear identical. As shown, the field penetration is not affected by the polymer evidencing the fact that fibrosis severely affects the performance of cochlear implant electrodes both functionally as well as driving up the system power consumption.

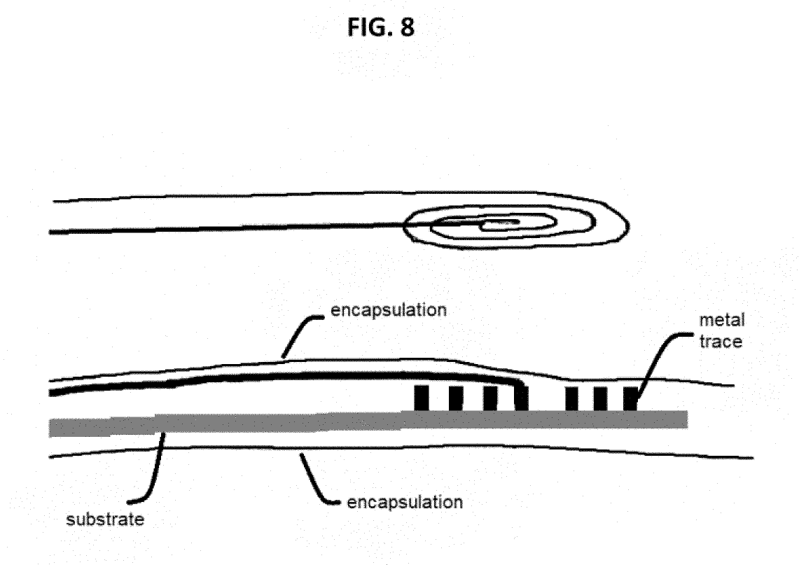

Turning to FIG. 8, an example view is shown looking down on an example coil that is encapsulated and used with a loop of one example implant system of this disclosure. Recent advances in 3D printed and flexible electronics for health and wellness provide an opportunity to greatly transform fabrication of neural interface materials. 3-D printed coils are constructed in the following exemplary manner. In a first example, a flexible material, e.g., polyimide or Kapton, serves as the substrate material. Conductive inks such as silver are printed on the substrate with either an inkjet printer or an aerosol printer. In this example, the coil can be made by 3-D printing with conductive inks, e.g., aluminum, silver, gold, copper. Use of an aerosol printer provides an advantage of reduce feature sizes down to 20-microns. Inkjet printers can approach 50-microns.

The coil is encapsulated along the outer surface and a substrate is disposed therein. On a distal end of the substrate are an array of metal traces that can be printed thereon. In particular, a cross section of the example coil is shown. The metal traces can be 15 to 50 microns wide and tall. The printed ink, or trace, carries the applied electrical current and may be shaped in the form of a coil (spiral) or even a simple loop. The number of turns on the coil serves to modulate the strength of the induced electric field. A return trace is printed onto an insulating bridge material to provide a return path for the current. In a final step, the coil, or array of coils, is fully encapsulated in biocompatible polymer such as Parylene-C. Formulations for inkjet cartridges or aerosol printers may be used.

As various changes can be made in the above-described subject matter without departing from the scope and spirit of the present disclosure, it is intended that all subject matter contained in the above description, or defined in the appended claims, be interpreted as descriptive and illustrative of the present disclosure. Many modifications and variations of the present disclosure are possible in light of the above teachings. Accordingly, the present description is intended to embrace all such alternatives, modifications, and variances which fall within the scope of the appended claims.

All patents, applications, publications, test methods, literature, and other materials cited herein are hereby incorporated by reference in their entirety as if physically present in this specification.

### REFERENCES

1. J. A. Bierer, and L. Litvak, “Reducing channel interaction through cochlear implant programming may improve speech perception: current focusing and channel deactivation,” Trends in Hearing, vol. 20, no, pp. 1-12, 2016.
2. B. Bonham and L. Litvak, “Current Focusing and Steering: Modeling, Physiology, and Psychophysics,” Hearing Research, vol. 242, no. 1-2, pp. 141-153, August 2008.
3. J. Choi, Y. Xue, W. Xia, T. R. Ray, J. T. Reeder, A. J. Bandodkar, D. Kang, S. Xu, Y. Huang and J. A. Rogers, “Soft, skin-mounted microfluidic systems for measuring secretory fluidic pressures generated at the surface of the skin by eccrine sweat glands,” Lab on a Chip, vol. 17, pp. 2572-2580, 2017.
4. G. M. Clark, J. Clark, T. Cardamone, M. Clarke, P. Nielsen, R. Jones R, B. Arhatari, N. Birbilis, R. Curtain, J. Xu, S. Wagstaff, P. Gibson, S. O'Leary, and J. Furness, “Biomedical studies on temporal bones of the first multi-channel cochlear implant patient at the University of Melbourne,” Cochlear Implants International, col. 15, up 2, pp. S1-S15, 2014.
5. A. Dhanasingh and C. Jolly, “An overview of cochlear implant electrode array designs,” Hearing Research, in press, (available at: https://doi.org/10.1016/j.heares.2017.10.005).
6. B. Escude, C. James, O. Deguine, N. Cochard, E. Eter, and B. Fraysse, “The size of the cochlea and predictions of insertion depth angles for cochlear implant electrodes,” Audiol Neurootol, vol. 11, pp. 27-33. 2006.
7. C. C. Finley, T. A. Holden, L. K. Holden, B. R. Whiting, R. A. Chole, G. J. Neely, T. E. Hullar, M. W. Skinner, “Role of electrode placement as a contributor to variability in cochlear implant outcomes,” Otol Neurotol, vol. 29, pp. 920-928, 2008.
8. J. B. Firszt, D. B. Koch, M. Downing, and L. Litvak, “Current steering creates additional pitch percepts in adult cochlear implant recipients,” Otology & Neurotology, vol. 28, no. 5, pp. 623-636, August 2007.
9. D. S. Lazard, A. L. Giraud, D. Gnansia, B. Meyer, and O. Sterkers, “Understanding the deafened brain: Implications for cochlear implant rehabilitation,” European Annals of Otorhinolaryngology, Head and Neck Diseases, vol. 129, no. 2, pp. 98-103, 2012.
10. L. Litvak, A. Spahr, and G. Emadi, “Loudness growth observed under partially tripolar stimulation: model and data from cochlear implant listeners,” Journal of Acoustical Society of America, vol. 122, no. 2, pp. 967-981, August 2007.
11. S. W. Lee, and S. I. Fried, “Suppression of subthalamic nucleus activity by micromagnetic stimulation,” IEEE Transactions on Neural Systems and Rehabilitation Engineering, vol. 23, no. 1, pp. 116-127, 2015.
12. R. T. Miyamoto, M. J. Hay-Mccutcheon, K. I. Kirk, D. M. Houston and T. Bergeson-Dana, “Language Skills of Profoundly Deaf Children Who Received Cochlear Implants Under 12 Months of Age: A Preliminary Study,” Acta Oto-laryngologica, vol. 128, no. 4, pp. 373-377, 2008.
13. D. R. Moore and R. V. Shannon, “Beyond Cochlear Implants, Awakening the Deafened Brain,” Nature Neuroscience, vol. 12, pp. 689-691, 2009.
14. S. A. Nauroze, J. Hester, B. Tehrani, W. Su, J. Bito, R. A. Bahr, J. Kimionis, and M. Tentzeris, “Additively manufactured RF components and modules: toward empowering the birth of cost-Efficient dense and ubiquitous IoT implementations,” Proceedings of the IEEE, vol. 105, no. 4, pp. 702-722, April 2017.
15. K. Nie, A. Barco, and F. Zheng, “Spectral and Temporal Cues in Cochlear Implant Speech Perception,” Ear & Hearing, vol. 27, pp. 208-207, 2006.
16. J. A. Rogers, T. Someya, and Y. Huang, “Materials and mechanics for stretchable electronics,” Science, vol. 327, no. 5973, March 2010.
17. Blake S. Wilson, “Getting a decent (but sparse) signal to the brain for users of cochlear implants,” Hearing Research, vol. 322, pp. 24-38, 2015.
18. J. Wouters, and J. H. McDermott, “Sound coding in cochlear implants, from electrical pulses to hearing,” IEEE Signal Processing Magazine, vol. 32, no. 2, pp. 67-80, 2015.

What is claimed is:

1. An implant system for stimulating a cochlea and/or a vestibular system, comprising:
a loop comprising a first end and a second end;
a wire comprising a first end connected to the first end of the loop and a second end connected to the second end of the loop; and
an electrode array disposed on or in communication with the wire, the electrode array adapted to be inserted into the cochlea;
wherein the first end of the wire receives electrical impulses representing recorded signals and conveys the electrical impulses through the loop towards the second end of the wire causing a time-varying magnetic field to be induced in the loop that creates a transient electrical field capable of stimulating the cochlea and/or vestibular system.

2. The system of claim 1, further comprising:
an insulator that encapsulates the loop.

3. The system of claim 2, wherein the insulator is a biocompatible polymer that encapsulates the antenna from between the first and second ends of the loop.

4. The system of claim 1, wherein the loop is a piece of metallic wire that is an inductive coil comprising the electrode array or in communication with the electrode array.

5. The system of claim 1, wherein stimulation of the cochlea by the transient electrical field causes an auditory brainstem response.

6. The system of claim 5, wherein the auditory brainstem response is indicative of a frequency of the sound signals represented by the received electrical impulses.

7. The system of claim 1, wherein stimulation of the vestibular system by the transient electrical field causes a vestibular nerve response.

8. The system of claim 7, wherein the vestibular nerve response is indicative of spatial and orientation information of the signals represented by the received electrical impulses.

9. The system of claim 1, further comprising:
a silicone plug surrounding a coil connected to or in communication with the loop to facilitate insertion of the loop inside the cochlea.

10. A method for stimulating a cochlea and/or a vestibular system, comprising:
delivering an implant system to the cochlea, the implant system comprising:
an electrode array adapted to be inserted into the cochlea;
a loop comprising a first end and a second;
a wire comprising a first end connected to the first end of the loop; and
a second end connected to the second end of the loop a second end, the implant system receiving electrical impulses representing sound signals;
conveying the electrical impulses through the loop to the electrode array inserted within the cochlea; and
inducing a time-varying magnetic field in the loop in response to the electrical impulses to create a transient electrical field that stimulates the cochlea and/or the vestibular system.

11. The method of claim 10, wherein stimulating of the cochlea and/or the vestibular system by the transient electrical field causes an auditory brainstem response and/or a vestibular nerve response.

12. The method of claim 11, wherein the auditory brainstem response is indicative of a frequency of the sound signals represented by the received electrical impulses.

13. The method of claim 11, wherein the vestibular nerve response is indicative of spatial and orientation information of the signals represented by the received electrical impulses.

14. The method of claim 10, wherein stimulation of the cochlea and/or the vestibular system by the transient electrical field varies depending upon the strength and timing of the received electrical impulses.

15. The method of claim 10, further comprising:
arranging the loop so as to not be in direct contact with tissue of the cochlea.

16. A method of fabricating a cochlear implant system, the method comprising:
forming a substrate with a flexible material;
printing metal traces on the substrate using conductive ink to form an array of coils;
separating each coil by a predetermined distance;
positioning insulating bridge material over the array of coils; and
encapsulating the substrate and the array of coils in a biocompatible polymer.

17. The method of claim 16, wherein the flexible material is polyimide or Kapton.

18. The method of claim 16, wherein the conductive ink is aluminum, silver, gold or copper.

19. The method of claim 16, wherein the step of printing is 3D printing.

20. The method of claim 16, wherein the predetermined distance is 5 mm.
